(19) United States
(12) Patent Application Publication
(10) Pub. No.: US 2020/0038822 A1
(43) Pub. Date: Feb. 6, 2020

(54) MODIFIED CONTROLLED CAVITATION FOR COSMETICS AND MEDICINAL DRUGS IN MANUFACTURING ENVIRONMENTS

(71) Applicant: Anwar A. Mohammed, San Jose, CA (US)

(72) Inventor: Anwar A. Mohammed, San Jose, CA (US)

(21) Appl. No.: 16/529,374

(22) Filed: Aug. 1, 2019

Related U.S. Application Data
(60) Provisional application No. 62/714,073, filed on Aug. 2, 2018.

(51) Int. Cl. B01F 11/02; A61K 9/51; A61K 8/02

(52) U.S. Cl. CPC B01F 11/0208; A61K 9/5192; B01F 2215/0032; B01F 2215/0031; A61K 8/0204

(57) ABSTRACT

A mechanism is described for facilitating controlled cavitation of medicines and cosmetics. A method of embodiments, as described herein, includes facilitating, by one or more processors of a controlled cavitation device, controlled cavitation dispersion for de-agglomeration of a compound that is agglomerated and represents a mixture of ingredients associated with a medical drug or a cosmetic item. The method may further include generating the medical drug or the cosmetic item based on the controlled cavitation dispersion of the compound.

### CLAIM OF PRIORITY

This application claims the benefit of and priority to U.S. Provisional Patent Application No. 62/714,073, by Dr. Anwar A. Mohammed, filed Aug. 2, 2018, entitled MANUFACTURING OF COSMETICS AND DRUGS LEVERAGED BY ADVANCED DISPERSIVE TECHNOLOGIES TO ENHANCE THEIR PERFORMANCE, the entire contents of which are incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD

Embodiments described herein generally relate to devices. More particularly, embodiments relate to facilitating controlled cavitation for cosmetics and medicinal drugs in manufacturing environment.

### BACKGROUND

Nano materials are used extensively for stretching the technological boundaries of cosmetics and medicinal drugs (“medicine” or simply “drugs”). Along with their physical quality that allows for easy formation of suspensions, these tiny/nano structures are known to exhibit a relatively greater surface area which can significantly enhance their behavior like strength, reactivity, electrical and chemical properties etc.

### BRIEF DESCRIPTION OF THE DRAWINGS

Embodiments are illustrated by way of example, and not by way of limitation, in the figures of the accompanying drawings in which like reference numerals refer to similar elements.

FIG. 1 a computing device hosting controlled-cavitation mechanism according to one embodiment.

FIG. 2 illustrates the controlled cavitation mechanism of FIG. 1 according to one embodiment.

FIG. 3A illustrates a modified controlled cavitation 300 according to one embodiment.

FIG. 3B illustrates a modified controlled cavitation dispersion transaction sequence according to one embodiment.

FIG. 3C illustrates an emulsion transaction sequence according to one embodiment.

FIG. 3D illustrates a microscopic view of drop size distribution according to one embodiment.

FIG. 4 illustrates a method for controlled cavitation in manufacturing of medicines and/or cosmetics according to one embodiment.

FIG. 5 illustrates an embodiment of an exemplary computing architecture that may be suitable for implementing various embodiments in accordance with some examples.

### DETAILED DESCRIPTION

In the following description, numerous specific details are set forth. However, embodiments, as described herein, may be practiced without these specific details. In other instances, well-known circuits, structures and techniques have not been shown in detail in order not to obscure the understanding of this description.

Embodiments provide for a novel technique for facilitating controlled cavitation for cosmetics and medicinal drugs in manufacturing environment. In one embodiment, controlled cavitation is applied to formation of cosmetics and/or drugs through a regular processes where certain ingredients, such as nano particles, are mixed together in appropriate proportions and then added into a solvent of some form and, in some cases, undergoing treatment to enhance their mixing, like thermal or other stimulation. For example, this mixture may then be exposed to a mixing procedure, such as planetary mixing, ultrasonic mixing, 3 ball milling, etc. Upon the mixture reaching the right rheology, it then undergoes a special cavitation dispersion process for deagglomeration.

It is contemplated that embodiments are not limited to any number or types of processes, materials, apparatus, or techniques for achieving the novel technique for controlled cavitation as it applies to cosmetics and drugs in various manufacturing environments.

It is further contemplated that “cosmetics”, as discussed throughout this document, refers to chemical compounds that are made available in the form of various materials, products, substances, etc., that are typically used to better a person's appearance. Cosmetics can mildly or radically enhance or alter a person's face or body, skin texture, scent or fragrance, etc. Cosmetics include (but are not limited to) creams, lipsticks, lotions, hair products, cleansers, etc.

Similarly, “drugs”, as discussed throughout this document, refers to pharmaceutical drugs or medication used for curing, treating, prevent, or diagnosing a condition or disease in people, animals, etc. As with cosmetics, drugs come in many forms and intensity levels, such as ranging from medications for common cold or headaches to serious conditions like cancer, etc.

Throughout this document, terms like “logic”, “component”, “module”, “framework”, “engine”, “mechanism”, “technique”, and/or the like, may be referenced interchangeably and include, by way of example, software, hardware, and/or any combination of software and hardware, such as firmware. Further, any use of a particular brand, word, term, phrase, name, acronym, or the like, such as “cavitation”, “controlled cavitation”, “nano particles”, “mixing”, “treating”, “rheology”, “cosmetics”, “medicines”, “drugs”, “user”, “material”, “wireless”, “computing device”, “smartphone”, “tablet computer”, “software application”, “social and/or business networking applications or websites”, “website”, or “site”, and/or the like, should not be read to limit embodiments to software or devices that carry that label in products or in literature external to this document.

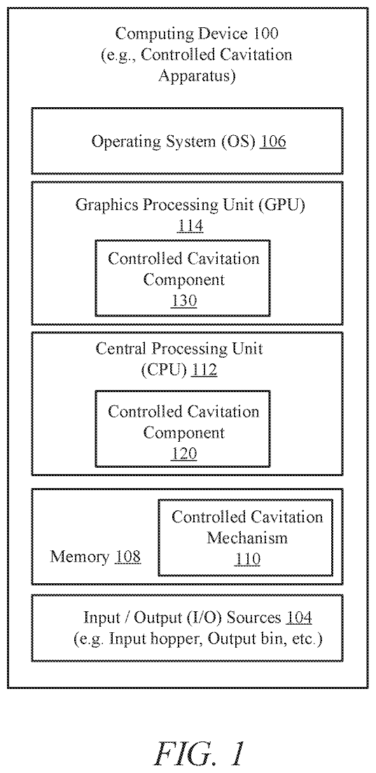

FIG. 1 illustrates a computing device 100 hosting controlled-cavitation mechanism 110 according to one embodiment. Computing or processing device (also referred to as “cavitation apparatus” or “cavitation system”) 100 represents a communication and data processing device for controlled cavitation with respect to cosmetics and medicine. Cavitation apparatus 100 represents a communication and data processing device for facilitating or performing controlled cavitation with respect to cosmetics, drugs, etc. It is contemplated and as will be further discussed later in this document, cavitation apparatus 100 may include any number and type of other parts or components, such as hopper, belt, shaker, etc., necessitated for performing controlled cavitation.

Further, cavitation apparatus 100 may include or coupled to or be associated with or facilitate by one or more of (but not limited to) smart voice command devices, intelligent personal assistants, home/office automation system, home appliances (e.g., washing machines, television sets, etc.), mobile devices (e.g., smartphones, tablet computers, etc.), gaming devices, handheld devices, wearable devices (e.g., smartwatches, smart bracelets, etc.), virtual reality (VR) devices, head-mounted displays (HMDs), Internet of Things (IoT) devices, laptop computers, desktop computers, server computers, set-top boxes (e.g., Internet-based cable television set-top boxes, etc.), global positioning system (GPS)-based devices, automotive infotainment devices, etc.

Further, cavitation apparatus 100 may include or coupled to or be associated with or facilitate by one or more of any number and type of other smart devices, such as (but not limited to) autonomous machines or artificially intelligent agents, such as a mechanical agents or machines, electronics agents or machines, virtual agents or machines, electro-mechanical agents or machines, etc. Examples of autonomous machines or artificially intelligent agents may include (without limitation) robots, autonomous vehicles (e.g., self-driving cars, self-flying planes, self-sailing boats, etc.), autonomous equipment (self-operating construction vehicles, self-operating medical equipment, etc.), and/or the like. Further, “autonomous vehicles” are not limited to automobiles but that they may include any number and type of autonomous machines, such as robots, autonomous equipment, household autonomous devices, and/or the like, and any one or more tasks or operations relating to such autonomous machines may be interchangeably referenced with autonomous driving.

Further, for example, cavitation apparatus 100 may include a computer platform hosting an integrated circuit (“IC”), such as a system on a chip (“SoC” or “SOC”), integrating various hardware and/or software components of cavitation apparatus 100 on a single chip. For example, cavitation apparatus 100 comprises a data processing device having one or more processors including (but not limited to) central processing unit 112 and graphics processing unit 114 that are co-located on a common semiconductor package.

As illustrated, in one embodiment, cavitation apparatus 100 may include any number and type of hardware and/or software components, such as (without limitation) graphics processing unit (“GPU” or simply “graphics processor”) 114, graphics driver (also referred to as “GPU driver”, “graphics driver logic”, “driver logic”, user-mode driver (UMD), UMD, user-mode driver framework (UMDF), UMDF, or simply “driver”) 116, central processing unit (“CPU” or simply “application processor”) 112, memory 104, network devices, drivers, and/or the like, as well as input/output (I/O) source(s) 108, such as touchscreens, touch panels, touch pads, virtual or regular keyboards, virtual or regular mice, ports, connectors, etc. Cavitation apparatus 100 may include operating system (OS) 106 serving as an interface between hardware and/or physical resources of the cavitation apparatus 100 and a user.

It is to be appreciated that a lesser or more equipped system than the example described above may be preferred for certain implementations. Therefore, any configuration of cavitation apparatus 100 may vary from implementation to implementation depending upon numerous factors, such as price constraints, performance requirements, technological improvements, or other circumstances.

Embodiments may be implemented as any or a combination of: one or more microchips or integrated circuits interconnected using a parentboard, hardwired logic, software stored by a memory device and executed by a microprocessor, firmware, an application specific integrated circuit (ASIC), and/or a field programmable gate array (FPGA). Terms like “logic”, “module”, “component”, “engine”, “circuitry”, “element”, and “mechanism” may include, by way of example, software, hardware, firmware, and/or a combination thereof.

In one embodiment, as illustrated, controlled-cavitation mechanism 110 may be hosted by memory 108 in communication with I/O source(s) 104, such as microphones, speakers, etc., of cavitation apparatus 100. In another embodiment, controlled-cavitation mechanism 110 may be part of or hosted by operating system 106. In yet another embodiment, controlled-cavitation mechanism 110 may be hosted or facilitated by graphics driver 116. In yet another embodiment, controlled-cavitation mechanism 110 may be hosted by or embedded in central processing unit (“CPU” or simply “application processor”) 112 and/or graphics processing unit (“GPU” or simply graphics processor”) 114 as one or more hardware components, such as controlled-cavitation component 120 at application processor 112, and/or controlled-cavitation component 130 at graphics processor 114.

For example, controlled-cavitation components 120, 130 may be implemented as or using one or more analog or digital circuits, logic circuits, programmable processors, programmable controllers, GPUs, digital signal processors (DSPs), application specific integrated circuits (ASICs), programmable logic devices (PLDs), field programmable logic devices (FPLDs), and/or the like. It is, therefore, contemplated that one or more portions or components of controlled-cavitation mechanism 110 may be employed or implemented as hardware, software, firmware, or any combination thereof.

In some embodiments, cavitation apparatus 100 includes a smart material handling component (“material component”) representing a hardware or firmware component hosted by one or more of application and graphics processors 112, 114. As will be further discussed in this document, in one embodiment, material component is facilitated by controlled-cavitation mechanism 110 to perform one or more novels tasks as described throughout this document.

As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events

Cavitation apparatus 100 may host network interface device(s) to provide access to a network, such as a LAN, a wide area network (WAN), a metropolitan area network (MAN), a personal area network (PAN), Bluetooth, a cloud network, a mobile network (e.g., 3^{rd} Generation (3G), 4^{th} Generation (4G), etc.), an intranet, the Internet, etc. Network interface(s) may include, for example, a wireless network interface having antenna, which may represent one or more antenna(e). Network interface(s) may also include, for example, a wired network interface to communicate with remote devices via network cable, which may be, for example, an Ethernet cable, a coaxial cable, a fiber optic cable, a serial cable, or a parallel cable.

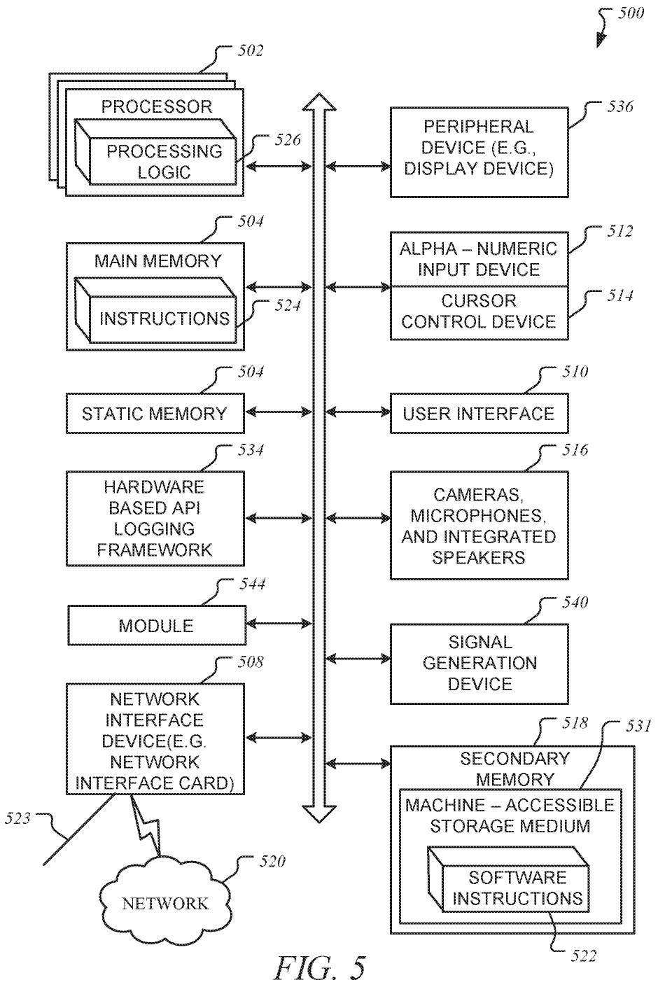

Embodiments may be provided, for example, as a computer program product which may include one or more machine-readable media having stored thereon machine-executable instructions that, when executed by one or more machines such as a computer, a data processing machine, a data processing device, network of computers, or other electronic devices, may result in the one or more machines carrying out operations in accordance with embodiments described herein. As further described with reference to processing architecture 500 of FIG. 5, a machine may include one or more processors, such as a CPU, a GPU, etc. A machine-readable medium may include, but is not limited to, floppy diskettes, optical disks, Compact Disc-Read Only Memories (CD-ROMs), magneto-optical disks, ROMs, Random Access Memories (RAMs), Erasable Programmable Read Only Memories (EPROMs), Electrically Erasable Programmable Read Only Memories (EEPROMs), magnetic or optical cards, flash memory, or other type of media/machine-readable medium suitable for storing machine-executable instructions.

For example, when reading any of the apparatus, method, or system claims of this patent to cover a purely software and/or firmware implementation, instructions associated with controlled-cavitation mechanism 110 may be expressly stored at a non-transitory computer readable storage device or storage disk such as a memory, a digital versatile disk (DVD), a compact disk (CD), a Blu-ray disk, etc., including the software and/or firmware.

Moreover, one or more elements of controlled-cavitation mechanism 110 may be downloaded as a computer program product, wherein the program may be transferred from a remote computer (e.g., a server) to a requesting computer (e.g., a client) by way of one or more data signals embodied in and/or modulated by a carrier wave or other propagation medium via a communication link (e.g., a modem and/or network connection).

Throughout this document, the term “user” may be interchangeably referred to as “viewer”, “observer”, “speaker”, “person”, “individual”, “end-user”, “developer”, “programmer”, “administrators”, and/or the like. For example, in some cases, a user may refer to an end-user, such as a consumer accessing a client computing device, while, in some other cases, a user may include a developer, a programmer, a system administrator, etc., accessing a workstation serving as a client computing device. It is to be noted that throughout this document, terms like “graphics domain” may be referenced interchangeably with “graphics processing unit”, “graphics processor”, or simply “GPU”; similarly, “CPU domain” or “host domain” may be referenced interchangeably with “computer processing unit”, “application processor”, or simply “CPU”.

It is to be noted that terms like “node”, “computing node”, “server”, “server device”, “cloud computer”, “cloud server”, “cloud server computer”, “machine”, “host machine”, “device”, “computing device”, “computer”, “computing system”, and the like, may be used interchangeably throughout this document. It is to be further noted that terms like “application”, “software application”, “program”, “software program”, “package”, “software package”, and the like, may be used interchangeably throughout this document.

Further, throughout this document, terms like “request”, “query”, “job”, “work”, “work item”, and “workload” are referenced interchangeably. Similarly, an “application” or “agent” may refer to or include a computer program, a software application, a game, a workstation application, etc., offered through an application programming interface (API), such as a free rendering API, such as Open Graphics Library (OpenGL®), DirectX® 11, DirectX® 12, etc., where “dispatch” may be interchangeably referenced as “work unit” or “draw”, while “application” may be interchangeably referred to as “workflow” or simply “agent”.

In some embodiments, terms like “display screen” and “display surface” may be used interchangeably referring to the visible portion of a display device while the rest of the display device may be embedded into a computing device, such as a smartphone, a wearable device, etc. It is contemplated and to be noted that embodiments are not limited to any particular computing device, software application, hardware component, display device, display screen or surface, protocol, standard, etc. For example, embodiments may be applied to and used with any number and type of real-time applications on any number and type of computers, such as desktops, laptops, tablet computers, smartphones, head-mounted displays and other wearable devices, and/or the like. Further, for example, rendering scenarios for efficient performance using this novel technique may range from simple scenarios, such as desktop compositing, to complex scenarios, such as three-dimensional (3D) games, augmented reality applications, etc.

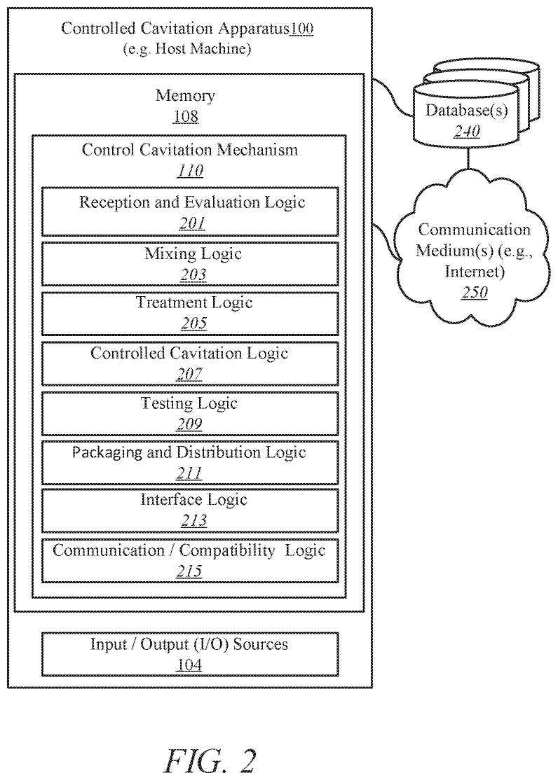

FIG. 2 illustrates controlled-cavitation mechanism 110 of FIG. 1 according to one embodiment. For brevity, many of the details already discussed with reference to FIG. 1 are not repeated or discussed hereafter. Further, for brevity and clarity, many of the well-known processes and components associated with general/generic type of cavitation are not discussed in this document. In one embodiment, controlled-cavitation mechanism 110 may include any number and type of components, such as (without limitations): reception and evaluation logic 201; mixing logic 203; treatment logic 205; controlled cavitation logic 207; testing logic 209; packaging and distribution logic 211; interface logic 213; and communication/compatibility logic 215. Controlled cavitation apparatus 100 is further shown as being in communication with one or more databases 240 and communication medium 250 (e.g., networks, such as cloud network, Internet, proximity network, etc.). For example, controlled cavitation apparatus 100 may include a 3D printer or be in communication with one or more 3D printers directly or over communication medium(s) 250.

In one embodiment, interface logic 213 may be used to offer user interface to allow the user to input request, track the progress of the request, and anticipate outputs, change or edit previously-submitted requests, etc., through one or more user interfaces offered by one or more display screens or devices that are part of or in communication with controlled cavitation apparatus 100.

As previously discussed, nano techniques and materials are used in manufacturing of various products for the flexibility they offer through the physical qualities, greater surface areas, etc., for enhancing behaviors like strength, reactivity, magnetic, optical, medicinal, aesthetics, electrical and chemical properties. However, using nano techniques in conventional techniques can be challenging in that they often end up flocculating and clumping, etc. When that happens, one of the best advantages of nano techniques, such as the creation of high surface area, is compromised, while the resultant performance is marginalized.

Embodiments provide for a novel technique for employing an advance nano dispersive technology, called controlled cavitation, as facilitated by controlled-cavitation mechanism 110 and/or one or more of controlled-cavitation components 120, 130, to ensure that nano materials undergo minimal flocculation/agglomeration, while performing at a significantly enhanced level. The higher surface area allows for superior delivery and efficacy of cosmetics and drugs.

In one embodiment, as facilitated by controlled-cavitated mechanism 110 and/or one or more of controlled-cavitation components 120, 130, controlled cavitation, which represents a nano dispersion technology, is used to ensure that most nano materials are in suspension within products, such as cosmetics, drugs, etc., remain un-agglomerated resulting in enhanced properties not available with other conventional techniques.

Cavitation

Cavitation refers to a dispersion process in which rapid pressure changes in dispersed liquid create formation of small vapor-filled cavities where the pressure is relatively low. Further, under higher pressure, these cavities, such as bubbles or voids, collapse while generating an intense shock wave causing dispersion. Nano materials are used extensively in medicine in, for example, preparation of tumor vaccines designed attack cancerous tissues. Nano materials are also used extensively for cosmetics, such as using nano zinc material for ultra-violet (UV) blocking sunscreen lotions.

Modified Cavitation

This disclosure offers a novel technique for nano dispersive technology of modified controlled cavitation (also referred to as “novel controlled cavitation” or simply “controlled cavitation” throughout this document) applicable to manufacturing of medicines and cosmetics ensuring that the nano materials used in medicines and cosmetics undergo minimal flocculation/clumping and perform their functions at a significantly enhanced manner. The higher surface area enables superior delivery and efficacy for medicine and cosmetics. The novel technique is a special process compatible with ISO 9001 and ISO 13485 requirements for manufacturing medicines and ISO 22716 for manufacturing cosmetics.

This novel modified controlled cavitation technique offers a quality of deflocculation and uniform particle sizing not seen by other dispersion technologies used for manufacturing medicines and cosmetics. Uniform particle sizing enable higher functional loading, enabling improved efficacy and potency of the respective medicine or cosmetic.

The novel processes associated with the novel technique for modified controlled cavitation for manufacturing medicines and cosmetics passes the material being cavitated through selected, fine ceramic orifices and into an expanded reaction zone which generates cavitation. The formation, growth and the resultant implosive collapse of the vacuum bubbles releases tremendous localized energy that breaks down and deflocculates/separates the agglomerated particles. The ceramic orifices for the manufacture of medicines and cosmetics can range from 3 to 24 mils depending upon the viscosity and type of material being used for the medicine or cosmetic.

As aforesaid, modified controlled cavitation is a novel cavitation technique designed to manufacture medicines and cosmetics under pressures ranging from 100 psi to as high as 50 Kpsi and temperatures ranging from 30 to 60 degrees C. It uses fine ceramic reactor tubes that can be juxtaposed in various configurations to create the required back pressure. The back pressure needed depends on the material properties of the item being cavitated, like viscosity, rheology, and the amount of agglomeration. It is this back pressure that causes the vacuum bubbles to collapse and generate shock waves and liquid microjets that break the medicine or cosmetics into a fine primary particle, with no agglomeration or clumping which results into high surface area for the materials undergoing the special process.

Further, this novel technique allows for handling both low and high viscosity medicine or cosmetic materials ranging from 1 Kcps to 250 Kcps. These materials may or may not contain nano materials, while minimizing flocculation and enhancing uniform particle distribution sharply.

Further, this novel technique for using controlled cavitation in manufacturing of drugs and cosmetics can offer increased permeability and penetrate deeper into the skin, delivering nutrients and nano particles to deeper layers of skin cells. Cavitated and non-agglomerated nano materials exhibit a superior uniform particle sizing that can result in exceptionally smooth facial and body creams. Cavitated cosmetics can also influence the biocompatibility and anti-bacterial properties of various cosmetics and drugs.

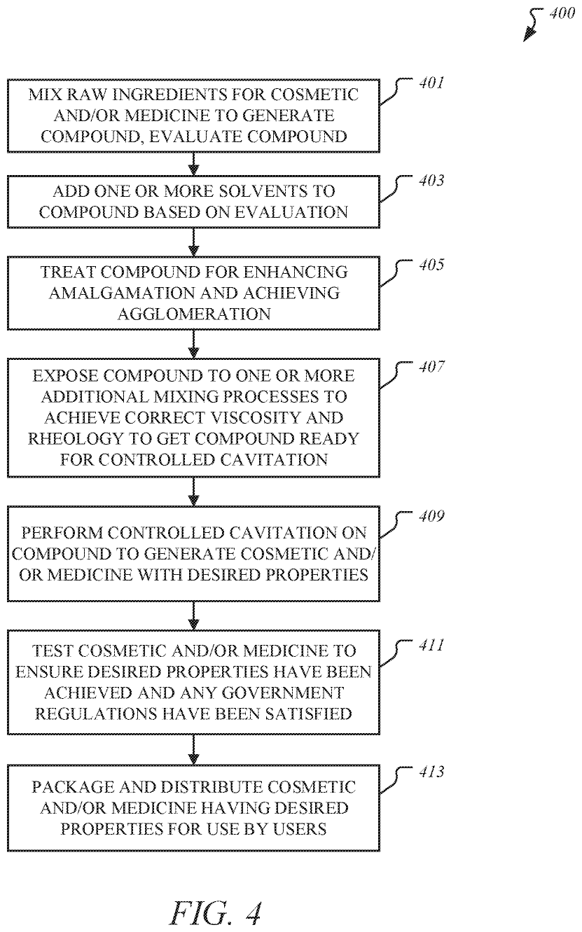

As further illustrated with reference to FIG. 4, controlled cavitation, as facilitated by controlled cavitation mechanism 110 and/or one or more controlled cavitation components 120, 130, is performed for cosmetics and drugs. For example, any formation of drugs or cosmetics undergo their normal production where various ingredients are first received and put through initial evaluation for authenticity and applicability as facilitated by reception and evaluation logic 201. It is contemplated that each drug or cosmetic requires different ingredients for preparation. For example, even two drugs with the same purpose, such as treatment for pain, could have different ingredients or quantities of even the same ingredients if, for example, they are designed to treat minor pain as opposed to major pain. Similar requirement and expectations are associated with cosmetics. Such ingredients may be received in a receiver, such as a hopper, associated with or that is part of cavitation apparatus 110 where the ingredients are then put through their initial evaluation for authenticity, quantity, and applicability.

Then, in one embodiment, the ingredients are mixed into a compound as facilitated by mixing logic 203. For example, this mixing may be performed in two stages, where at a first stage, the basic ingredients are mixed together to form a compound that is expected from the mixture of such ingredient and then, at a second stage, the compound is made whole and ready for subsequent processes by adding any solvents to the compound as facilitated by mixing logic 203.

Upon addition of the solvent and preparation of the compound for additional processes, the compound is then put through some treatment, such as thermal stimulation, mechanical stimulation, etc., to enhance amalgamation in the compound as facilitated by treatment logic 205. It is contemplated that may of these processes, such as mixing, treating, amalgamation, etc., are well known in the field of production and manufacturing of drugs and cosmetics and are beyond the scope of this invention. Therefore, for the sake of brevity and clarity, any such processes, systems, equipment, and apparatus are not discussed in detail in this document.

For example, upon treatment of the compound for the purposes of amalgamation as facilitated by treatment logic 205, the compound is then exposed to other forms of one or more mixing procedures, such planetary mixing, ultrasonic mixing, three ball milling, etc., as facilitated by mixing logic 203. This additional mixing of the compound is performed to achieve the correct viscosity and rheology to get the compound prepared for cavitation.

Upon having the compound reach the correct viscosity and rheology, in one embodiment, the compound is then put through a special dispersion process, called controlled cavitation, for de-agglomeration as facilitated by controlled cavitation logic 207. This controlled cavitation is performed near the end of the process to ensure de-agglomeration to generate enhanced material performance. For example, with controlled cavitation, rapid changes of pressure may be applied to this compound to form small vapor-filled cavities, where the pressure is comparatively low. The controlled cavitated compound is then tested for accuracy of the outcome, whether it be a final drug or a final cosmetic product as facilitated by testing logic 209. Upon testing and verifying the accuracy of the drug/cosmetic project, the final product is then packaged and shipped for distribution as facilitated by packaging and distribution logic 211.

Further, interface logic 213 may offer one or more user interfaces (e.g., Graphical User Interface (GUI), application-based interface, etc.) that the user may used to initiate and facilitate one or more processes discussed above and carry the processes through the entire line of processes, while adjusting quantities, speeds, and fixing any errors, etc., for a seamless and efficient flow of processes.

It is contemplated that embodiments are not limited to these processes and that some of the processes, such as treatment to enhance amalgamation or second level of mixing are not necessarily necessitated or simply replaced by a more enhanced controlled cavitation.

As previously described, cavitation refers to a process where rapid changes of pressure within a liquid can generate formation of small vapor-filled cavities, in areas where typically pressure is relatively low. When subjected to higher pressure, these cavities, called bubbles or voids, may collapse and generate intense shock wave. This novel controlled cavitation process for manufacturing medicine and cosmetics passes the material being cavitated through fine ceramic orifices and into an expanded reaction zone which generated cavitation. The formation, growth, and the resultant implosive collapse of the vacuum bubbles releases tremendous localized energy that breaks down and separates the agglomerated particles. The ceramic orifices can range from 4 to 20 mils depending upon the viscosity and type of material that is to be cavitated.

Further, this novel technique for controlled cavitation is designed to work under pressure as high as 40 Kpsi, which is relatively high. It uses fine ceramic reactor tubes that are juxtaposed in various configurations to create the necessary back pressure. This back pressure may dependent on various material properties of the item being cavitated, like viscosity, rheology, and the amount of agglomeration. Further, this back pressure may cause the vacuum bubbles to collapse and generate shock waves and liquid microjets that break everything into a fine primary particle, which no agglomeration or clumping that results in high surface area for those materials undergoing a controlled cavitation process.

In one embodiment, this novel technique for controlled cavitation is also regarded unique because it is capable of handling both low and high viscosity (>200 Kcps) materials that are used mainly for drugs and cosmetics, and which may or may not contain nano-materials, and minimize agglomeration and sharply enhance uniform particle distribution. This novel technique allows for using advanced nano dispersive technology, called controlled cavitation, applicable to manufacturing of medicines and cosmetics ensuring that any nano materials used in medicines and cosmetics undergo minimal agglomeration, while performing in a significantly enhanced manner. This higher surface area enables superior delivery and efficacy for drugs and cosmetics.

Further, controlled cavitation process is compatible with industry requirements for materials, processes, equipment, etc., such as ISO 9001, ISO 13485, etc., for manufacturing medicines and ISO 22716 for manufacturing cosmetics. Controlled cavitation further offers a quality of dispersion and uniform particle sizing not seen by other dispersion technologies used for manufacturing medicines and cosmetics, where uniform particle sizing enables higher functional loading allowing improved efficacy and potency of the respective drug or cosmetics.

Embodiments provide for a novel technique for controlled cavitation for drugs and cosmetics, where nano technology is used for enabling the creation of some power materials with unprecedented qualities. Nano materials are powerful in their application as they have been allowed to increase their surface to volume ratio significantly, while having comparatively much higher surface area. This higher surface area enables the nano materials to have dramatically improved qualities like chemical reactivity, electrical performance, optical enhancements, and much more. For example, regular silver melts at around 960 degrees Celsius, where nano silver can melt at temperatures below 160 degrees Celsius. This was unimaginable 20 years ago.

Nano materials agglomerate or form clumps very easily when they are mixed within a solvent. This debilitates and impairs their optimal performance significantly, where most nano materials are mixed with solvents for manufacturing. This novel technique for controlled cavitation process minimizes the formation of agglomerates and clumping, where this allows any nano materials to retain their high surface area which renders them the ability to exhibit vastly superior performance. Further, using this novel controlled cavitation process, drugs and cosmetics may be manufactured at pressures as high as 30 Kpsi and with a material exhibiting a viscosity as high as 180 Kcps. Cavitation is a dispersion process in which rapid pressure changes in dispersed liquid to create this formation of small vapor-filled cavities where the pressure is relatively low. Under higher pressure, these cavities, called bubbles or voids, collapse while generating intense shock waves causing very fine dispersion.

Controlled cavitation offers a quality of dispersion and uniform particle sizing not seen by other conventional dispersion techniques. This is relevant to and effective with both nano and non-nano materials. Uniform particle sizing enables higher functional loading allowing improved efficacy and potency of the respective drugs or cosmetics. So even if a medicine or cosmetic product does not have any nano materials, this novel technique enhances their performance by offering more potent materials that are also smooth to the touch. This is beneficial for both drugs and cosmetics.

Embodiments provide for a sophisticated manufacturing process for drugs and cosmetics, which leverages advanced nano dispersion technologies through controlled cavitation to create nano suspensions, which prevent or minimize the agglomeration of nano particles. This allows for enhance of performance of drugs and cosmetics. The higher surface area, caused by preventing the flocculation of the nano materials, enhances both the efficacy and delivery of drugs and cosmetics. Further, even without any nano materials, the fine particle distribution generated through this novel technique allow for higher filler loading and fine particle size distribution that is unmatched by other approaches. This fine loading results in enhanced efficacy, while this fine distribution results in much smoother cosmetics.

Embodiments allow for ensuring that nano materials do not flocculate/agglomerate, while maintaining the high surface to volume ratio yielding to a relatively higher surface area. This is regarded as an essential quality of a nano material. Essentially, this process significantly augments and enhances the performance of nano materials by ensuring a higher comparative surface area.

Controlled Cavitation Properties with Respect to Medicine

With respect to drugs, embodiments provide for this novel technique for employing controlled cavitation to allow for pharmaceutical and cosmetic industries to use nano materials to perform at their optimal efficiency, while minimizing deflocculation, agglomeration and clumping. This allows the nano material to maintain a high surface area for offering increased potential for reactivity and potency that are not achievable with conventional non-cavitated materials (e.g., increased efficacy).

Further, cavitated materials can enhance any delivery of drugs through their enhanced reactivity by easily permeating through skin and other body tissues. This is increasingly effective with topically administered medicines, such as creams. Other drugs, like pain killers, asthma medications, etc., can benefit from maximum and faster efficacy by having nano materials that are non-agglomerated as opposed to agglomerated. Similarly, cavitated and non-agglomerated non materials may be used to lower the dosage of drugs (that is typically required by patients) by offering increased potency and reduced potential for side-effects. This is specifically beneficial for abrasive treatments, such as those used for serious illnesses like cancer.

Moreover, the novel techniques offer potential to elongate effects of drugs by combining the controlled cavitation technique with processes for prolonged or timed releases. For example, this increased duration is achieved through employing various processes, such as capsulizing medications, creating liposomal forms, etc. Due to their superior uniform particle sizing, cavitated drugs also offer exceptionally smooth medical ointments that can be comforting in special cases, such as when a patient is bed-ridden or wheal-chair-bound.

Even those drugs and cosmetics that do not use nano materials can be enhanced in one or more ways through this novel technique of controlled cavitation by offering a cavitation process that minimizing clumping and offers unmatched uniform particle sizing. For example, a cavitated drug offers improved precision for drug transportation and can be expanded to controlled time release, improved contrast medium, rapid medical testing, and manufacturing of advanced materials for implants and prostheses.

As described above, there are numerous drugs, such as pain killers, asthma medicine, cardiac medicine, etc., that could be enabled to work faster by having nano materials that are non-agglomerated as opposed to agglomerated. Further, cavitated and non-agglomerated nano materials can lower the dosage of various drugs having increased potency, such as for cancer treatments. Cavitated material can also enhance deliver of drugs due to their enhanced reactivity by permeating through skin and other body tissues relatively easier and faster. Moreover, because of their superior uniform particle sizing they can offer exceptionally smooth medical ointments that could be comforting in special cases, such as where a patent is bed-ridden or wheel-chair-bound. It is contemplated that even drugs and cosmetics that do not use nano materials can benefit from this novel controlled-cavitation apparatus and process because this novel technique will likely minimize clumping and offer unmatched uniform particle sizing. It is further contemplated that cavitated drugs can offer much improved selectivity for drug transportation and controlled release, improved contrast medium, and rapid medical testing, etc., along with helping manufacture advanced material for manufacturing of implants and prostheses.

Controlled Cavitation Properties with Respect to Cosmetics

This novel technique for using controlled cavitation for drugs and/or cosmetics allows for increased potency and delivery of cosmetic products, such as sunscreen lotion that uses nano zinc for UV blocking. This potency is further enhanced because of high surface areas that is created and the delivery that is enhanced through uniform particle sizing and minimization of clumping. Further, nano materials like zine oxide, titanium dioxide, tris-biphenyl triazine, etc., are used in cosmetics, while other nano materials that are used in cosmetics also include nanosomes, liposomes, fullerenes, solid lipid nanoparticles, etc. Further, cavitated and non-agglomerated cosmetics that use nano particles can offer increased permeability and penetrate deeper into the skin, delivering nutrients and nano particles to deeper layers of skin cells.

Cavitated and non-agglomerated nano materials exhibit a superior uniform particle sizing the results into exceptionally smooth facial and body creams. Cavitated nano-cosmetics exhibit enhanced optical properties attained at the nano level like color, shine, solubility, luminosity, fluorescence, thermochromocity, UV sensitivity, and transparency. Cavitated cosmetics can also influence the biocompatibility and anti-bacterial properties of any drug or cosmetic product. Controlled cavitation has the potential to increase efficacy, decrease onset time, and prolong duration of cosmetics.

Further, any use of a particular brand, word, term, phrase, name, and/or acronym, such as “nano material”, “cavitation”, “controlled cavitation”, “drug”, “medicine”, “cosmetics”, “agglomeration”, “amalgamation”, “clumping”, “mixing”, “solvent”, etc., should not be read to limit embodiments to software or devices that carry that label in products or in literature external to this document.

Communication/compatibility logic 215 may be used to facilitate the needed or desired communication and compatibility between any number of devices of and/or with controlled cavitation apparatus 100 and/or various components of controlled cavitation mechanism 100. Some of the devices may include client or server computing devices, other medicines and/or cosmetics manufacturing apparatus, input/output devices (e.g., cameras, sensors, detectors, microphones, speakers, display devices, etc.), databases, networks, etc.

Communication/compatibility logic 215 may be used to facilitate dynamic communication and compatibility between various components, networks, database(s) 240, and/or communication medium(s) 250, etc., and any number and type of other computing devices (such as wearable computing devices, mobile computing devices, desktop computers, server computing devices, etc.), processing devices (e.g., central processing unit (CPU), graphics processing unit (GPU), etc.), capturing/sensing components (e.g., non-visual data sensors/detectors, such as audio sensors, olfactory sensors, haptic sensors, signal sensors, vibration sensors, chemicals detectors, radio wave detectors, force sensors, weather/temperature sensors, body/biometric sensors, scanners, etc., and visual data sensors/detectors, such as cameras, etc.), user/context-awareness components and/or identification/verification sensors/devices (such as biometric sensors/detectors, scanners, etc.), memory or storage devices, data sources, and/or database(s) (such as data storage devices, hard drives, solid-state drives, hard disks, memory cards or devices, memory circuits, etc.), network(s) (e.g., Cloud network, Internet, Internet of Things, intranet, cellular network, proximity networks, such as Bluetooth, Bluetooth low energy (BLE), Bluetooth Smart, Wi-Fi proximity, Radio Frequency Identification, Near Field Communication, Body Area Network, etc.), wireless or wired communications and relevant protocols (e.g., Wi-Fi®, WiMAX, Ethernet, etc.), connectivity and location management techniques, software applications/websites, (e.g., social and/or business networking websites, business applications, games and other entertainment applications, etc.), programming languages, etc., while ensuring compatibility with changing technologies, parameters, protocols, standards, etc.

Controlled cavitation apparatus 100 may further provide a user interface (e.g., graphical user interface (GUI)-based user interface, Web browser, cloud-based platform user interface, software application-based user interface, other user or application programming interfaces (APIs), etc.) as facilitated by interface logic 213. Controlled cavitation apparatus 100 may further include I/O source(s) 104 having input component(s), such as camera(s) (e.g., Intel® RealSense™ camera), microphone(s), sensors, detectors, keyboards, mice, etc., and output component(s), such as display device(s) or simply display(s) (e.g., integral displays, tensor displays, projection screens, display screens, etc.), speaker devices(s) or simply speaker(s), etc.

Controlled cavitation apparatus 100 is further illustrated as having access to and/or being in communication with one or more database(s) 240 and/or one or more of other computing or printing devices over one or more communication medium(s) 250 (e.g., networks such as a proximity network, a cloud network, an intranet, the Internet, etc.).

In some embodiments, database(s) 240 may include one or more of storage mediums or devices, repositories, data sources, etc., having any amount and type of information, such as data, metadata, etc., relating to any number and type of applications, such as data and/or metadata relating to one or more users, physical locations or areas, applicable laws, policies and/or regulations, user preferences and/or profiles, security and/or authentication data, historical and/or preferred details, and/or the like.

As aforementioned, terms like “logic”, “module”, “component”, “engine”, “circuitry”, “element”, and “mechanism” may include, by way of example, software, hardware, firmware, and/or any combination thereof.

In one embodiment, I/O source(s) 104 may include one or more of containers, bins, hoppers, etc., to allow for reception and output of certain materials and objects. For example, reception and evaluation logic 201 may facilitate a hopper or a bin of I/O source(s) 104 to accept the necessary raw material typically required for manufacturing any number or type of medicines and cosmetics and then upon processing the material through controlled cavitation apparatus 100, as facilitated by controlled cavitation mechanism 110, the final product, such as a plate or a cup, etc., is outputted in a bit or an output container as facilitated by reception and evaluation logic 201.

I/O source(s) 104 may further include any number or type of microphone(s), camera(s), speaker(s), display(s), etc., for capture or presentation of data. For example, as facilitated by reception and evaluation logic 201, one or more of microphone(s) may be used to detect speech or sound simultaneously from users, such as speakers. Similarly, as facilitated by reception and evaluation logic 201, one or more of camera(s) may be used to capture images or videos of a geographic location (whether that be indoors or outdoors) and its associated contents (e.g., furniture, electronic devices, humans, animals, trees, mountains, etc.) and form a set of images or video streams.

Similarly, as illustrated, output component(s) may include any number and type of speaker(s) or speaker device(s) to serve as output devices for outputting or giving out audio from controlled cavitation apparatus 100 for any number or type of reasons, such as human hearing or consumption. For example, speaker(s) work the opposite of microphone(s) where speaker(s) convert electric signals into sound.

Moreover, input component(s) may include any number or type of cameras, such as depth-sensing cameras or capturing devices that are known for capturing still and/or video red-green-blue (RGB) and/or RGB-depth (RGB-D) images for media, such as personal media. Such images, having depth information, have been effectively used for various computer vision and computational photography effects, such as (without limitations) scene understanding, refocusing, composition, cinema-graphs, etc. Similarly, for example, displays may include any number and type of displays, such as integral displays, tensor displays, stereoscopic displays, etc., including (but not limited to) embedded or connected display screens, display devices, projectors, etc.

Input component(s) may further include one or more of vibration components, tactile components, conductance elements, biometric sensors, chemical detectors, signal detectors, electroencephalography, functional near-infrared spectroscopy, wave detectors, force sensors (e.g., accelerometers), illuminators, eye-tracking or gaze-tracking system, head-tracking system, etc., that may be used for capturing any amount and type of visual data, such as images (e.g., photos, videos, movies, audio/video streams, etc.), and non-visual data, such as audio streams or signals (e.g., sound, noise, vibration, ultrasound, etc.), radio waves (e.g., wireless signals, such as wireless signals having data, metadata, signs, etc.), chemical changes or properties (e.g., humidity, body temperature, etc.), biometric readings (e.g., figure prints, etc.), brainwaves, brain circulation, environmental/weather conditions, maps, etc. It is contemplated that “sensor” and “detector” may be referenced interchangeably throughout this document. It is further contemplated that one or more input component(s) may further include one or more of supporting or supplemental devices for capturing and/or sensing of data, such as illuminators (e.g., IR illuminator), light fixtures, generators, sound blockers, etc.

It is further contemplated that in one embodiment, input component(s) may include any number and type of context sensors (e.g., linear accelerometer) for sensing or detecting any number and type of contexts (e.g., estimating horizon, linear acceleration, etc., relating to a mobile computing device, etc.). For example, input component(s) may include any number and type of sensors, such as (without limitations): accelerometers (e.g., linear accelerometer to measure linear acceleration, etc.); inertial devices (e.g., inertial accelerometers, inertial gyroscopes, micro-electro-mechanical systems (MEMS) gyroscopes, inertial navigators, etc.); and gravity gradiometers to study and measure variations in gravitation acceleration due to gravity, etc.

Similarly, output component(s) may include dynamic tactile touch screens having tactile effectors as an example of presenting visualization of touch, where an embodiment of such may be ultrasonic generators that can send signals in space which, when reaching, for example, human fingers can cause tactile sensation or like feeling on the fingers. Further, for example and in one embodiment, output component(s) may include (without limitation) one or more of light sources, display devices and/or screens, audio speakers, tactile components, conductance elements, bone conducting speakers, olfactory or smell visual and/or non/visual presentation devices, haptic or touch visual and/or non-visual presentation devices, animation display devices, biometric display devices, X-ray display devices, high-resolution displays, high-dynamic range displays, multi-view displays, and head-mounted displays (HMDs) for at least one of virtual reality (VR) and augmented reality (AR), etc.

It is contemplated that embodiment are not limited to any number or type of use-case scenarios, architectural placements, or component setups; however, for the sake of brevity and clarity, illustrations and descriptions are offered and discussed throughout this document for exemplary purposes but that embodiments are not limited as such. Further, throughout this document, “user” may refer to someone having access to one or more computing devices, such as controlled cavitation apparatus 100, and may be referenced interchangeably with “person”, “individual”, “human”, “him”, “her”, “child”, “adult”, “viewer”, “player”, “gamer”, “developer”, programmer”, and/or the like.

Throughout this document, terms like “logic”, “component”, “module”, “framework”, “engine”, “tool”, “circuitry”, and/or the like, may be referenced interchangeably and include, by way of example, software, hardware, firmware, and/or any combination thereof. In one example, “logic” may refer to or include a software component that works with one or more of an operating system, a graphics driver, etc., of a computing device, such as controlled cavitation apparatus 100. In another example, “logic” may refer to or include a hardware component that is capable of being physically installed along with or as part of one or more system hardware elements, such as an application processor, a graphics processor, etc., of a computing device, such as controlled cavitation apparatus 100. In yet another embodiment, “logic” may refer to or include a firmware component that is capable of being part of system firmware, such as firmware of an application processor or a graphics processor, etc., of a computing device, such as controlled cavitation apparatus 100.

Further, any use of a particular brand, word, term, phrase, name, and/or acronym, such as “nano material”, “cavitation”, “controlled cavitation”, “drug”, “medicine”, “cosmetics”, “agglomeration”, “amalgamation”, “clumping”, “mixing”, “solvent”, “real-time”, “automatic”, “dynamic”, “user interface”, “camera”, “sensor”, “microphone”, “display screen”, “speaker”, “verification”, “authentication”, “privacy”, “user”, “user profile”, “user preference”, “sender”, “receiver”, “personal device”, “smart device”, “mobile computer”, “wearable device”, “IoT device”, “proximity network”, “cloud network”, “server computer”, etc., should not be read to limit embodiments to software or devices that carry that label in products or in literature external to this document.

It is contemplated that any number and type of components may be added to and/or removed from controlled cavitation mechanism 110 and/or a hardware/firmware-based material component to facilitate various embodiments including adding, removing, and/or enhancing certain features. For brevity, clarity, and ease of understanding of controlled cavitation mechanism 110, many of the standard and/or known components, such as those of a computing device are not shown or discussed here. It is contemplated that embodiments, as described herein, are not limited to any technology, topology, system, architecture, and/or standard and are dynamic enough to adopt and adapt to any future changes.

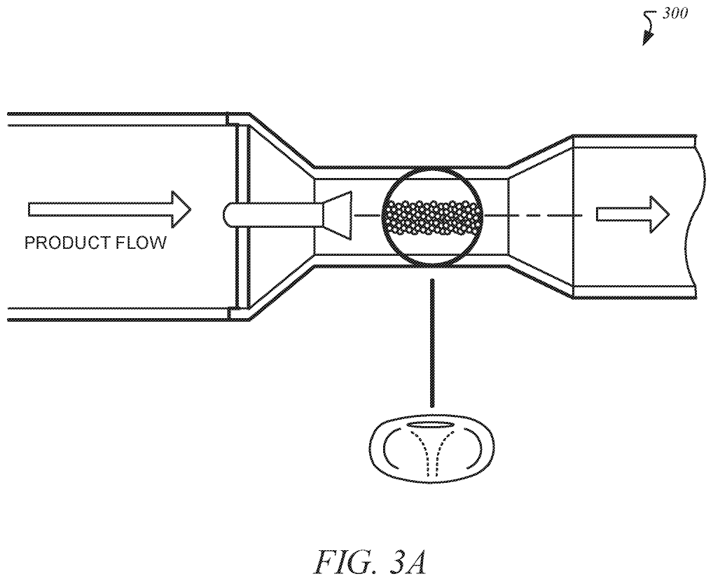

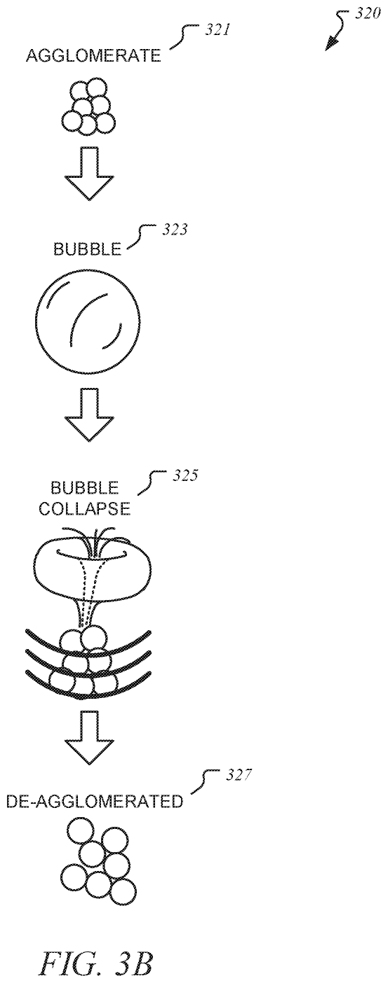

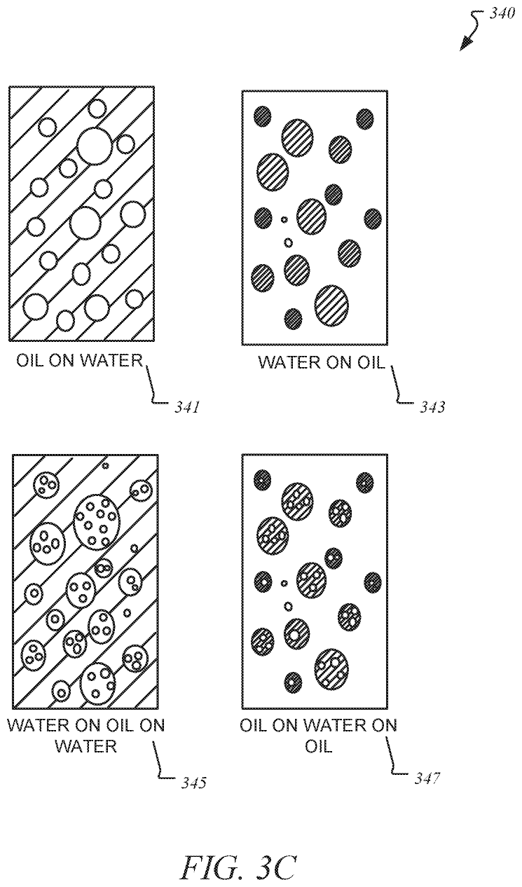

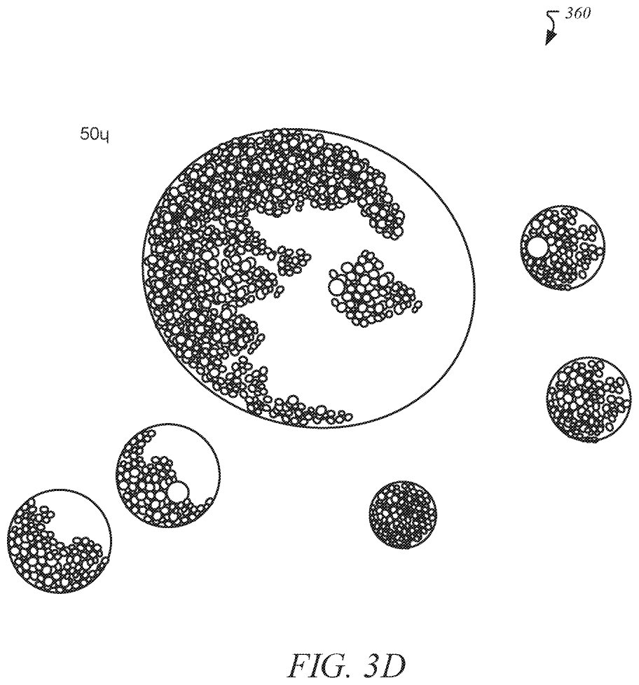

FIG. 3A illustrates a modified controlled cavitation (MCC) 300 according to one embodiment. For brevity, many of the details already discussed with reference to FIGS. 1-2 are not repeated or discussed hereafter. Further, for brevity, many of the known processes and components associated with cavitation are not discussed in this document. Moreover, embodiments are not limited to any type or order of placement of components or flow of processes and thus embodiments are not limited or restricted to the illustration of FIG. 2.

As previously discussed, cavitation refers to a physical phenomenon that generates creation, progression, and implosive collapse of vacuum and/or vapor bubbles in a liquid releasing tremendous localized energy. This novel technique for controlled cavitation allows for MCC 300 to work in close collaboration with supplies designed specifically for manufacturing of medicine and cosmetics materials and their formulations using both nano materials and micro structured materials.

As illustrated, MCC 300 refers to a modified controlled cavitation process that works on medicines and cosmetics due to its viscosity, rheology, and particle size distribution, high interfacial areas, between immiscible liquids, high surface areas, and other specific requirements inherent to manufacturing of such products. MCC 300 enables un-agglomerated materials, which means no clumping resulting into high surface areas which, in turns, offers high reactivity and enhanced performance and other advantages like uniform particles size distribution, unmatched dispersion of multiple ingredients, higher interfacial area between two immiscible fluids and many other advantages described elsewhere in this document.

FIG. 3B illustrates an MCC dispersion transaction sequence 320 according to one embodiment. For brevity, many of the details already discussed with reference to FIGS. 1-3A are not repeated or discussed hereafter. Further, for brevity, many of the known processes and components associated with cavitation are not discussed in this document. Moreover, embodiments are not limited to any type or order of placement of components or flow of processes and thus embodiments are not limited or restricted to the illustration of FIG. 3A.

As illustrated, MCC 300 of FIG. 3A effectively combines medium and high viscosity materials and generates extreme conditions required for de-agglomeration without altering the particle morphology of the cavitated material. These two qualities play a role in manufacturing of medicine and cosmetics as described throughout this document.

For example, transaction sequence 320 starts with agglomeration of a compound at 321, followed by bubble formation at 323. At 325, the bubble collapses and shockwaves are sent out, while the agglomerated particles are then de-agglomerated at block 327.

FIG. 3C illustrates an emulsion transaction sequence 340 according to one embodiment. For brevity, many of the details already discussed with reference to FIGS. 1-3B are not repeated or discussed hereafter. Further, for brevity, many of the known processes and components associated with cavitation are not discussed in this document. Moreover, embodiments are not limited to any type or order of placement of components or flow of processes and thus embodiments are not limited or restricted to the illustration of FIG. 3B.

In one embodiment, while manufacturing medicine or cosmetics, the management of emulsions plays a role in improving performance. For example, MCC 300 of FIG. 3A allows for management of emulsions used in medicine and cosmetics in a superior manner. Further, it allows for creation of high interfacial area between two fluids that are immiscible. This novel technique allows for generation of more uniform drop size and uniform drop size distribution that enhances product performance and minimizes the need for surfactants and emulsifiers.

As illustrated, transaction sequence 340 begins with oil on water at 341, followed by water on oil at 343. This is further followed by water on oil on water at 345 and then finally with oil on water on oil at 347.

FIG. 3D illustrates a microscopic view 360 of drop size distribution according to one embodiment. For brevity, many of the details already discussed with reference to FIGS. 1-3C are not repeated or discussed hereafter. Further, for brevity, many of the known processes and components associated with cavitation are not discussed in this document. Moreover, embodiments are not limited to any type or order of placement of components or flow of processes and thus embodiments are not limited or restricted to the illustration of FIG. 3C.

Based on the process and techniques of FIGS. 3A-3C, FIG. 3D illustrates microscopic view 360 of a drop-size distribution of the compound to manufacture medicines or cosmetics. The novel technique of controlled cavitation allows for high interfacial area between two fluids (e.g., oil, water) that are immiscible and enables generation of more uniform drop-size and uniform drop-size distribution 360 that enhances the product performance and minimizing the need for surfactants and emulsifiers.

FIG. 4 illustrates a method 400 for controlled cavitation for manufacturing of cosmetics and/or medical drugs according to one embodiment. For brevity, many of the details previously discussed with reference to FIGS. 1-3D may not be discussed or repeated hereafter. Further, for brevity, many of the known processes and components associated with cavitation processes and apparatus are not discussed in this document. Any processes relating to this method 400 may be performed by processing logic that may comprise hardware (e.g., circuitry, dedicated logic, programmable logic, etc.), software (such as instructions run on a processing device), or a combination thereof, as facilitated by controlled cavitation mechanism 110 and/or one or more of controlled cavitation components 120, 130 of FIG. 1. The processes associated with this method 400 may be illustrated or recited in linear sequences for brevity and clarity in presentation; however, it is contemplated that any number of them can be performed in parallel, asynchronously, or in different orders.

As described in detail with respect to FIG. 2, method 400 begins at block 401 with mixing and evaluating of essential ingredients necessary for manufacturing of medical drugs and/or cosmetics. Upon mixing, method 400 continues at block 403 with addition of one or more solvents to the mixed compound of the ingredients. At block 405, the compound is treated with to enhance amalgamation or agglomeration of the compound, where the compound is put through one or more treatments, such as thermal simulation, mechanical simulation, etc. At block 407, the amalgamated compound may then be put through other forms of mixing procedures, such as one or more of planetary mixing, ultrasonic mixing, 3 ball milling, etc., to correct viscosity and rheology to get the compound ready for controlled cavitation.

In one embodiment, once the compound has been mixed and made ready for controlled cavitation, at block 409, controlled cavitation is performed on the compound for de-agglomeration of the compound. At block 411, the de-agglomerated compound is then tested to confirm that the output is the expected drug and cosmetic with the desired properties. At block 413, the tested final product is then packaged and shipped out for use by users.

FIG. 5 illustrates a diagrammatic representation of a machine 500 in the exemplary form of a computer system, in accordance with one embodiment, within which a set of instructions, for causing machine 500 to perform any one or more of the methodologies discussed herein, may be executed. Machine 500 may be the same as or similar to or contained within or include printing apparatus 100 of FIG. 1 to perform or execute one or more methodologies discussed throughout this document. In alternative embodiments, machine 500 may be connected (e.g., networked) to other machines either directly, such as via media slot or over a network, such as a cloud-based network, a Local Area Network (LAN), a Wide Area Network (WAN), a Metropolitan Area Network (MAN), a Personal Area Network (PAN), an intranet, an extranet, or the Internet. The machine may operate in the capacity of a server or a client machine in a client-server network environment, or as a peer machine in a peer-to-peer (or distributed) network environment or as a server or series of servers within an on-demand service environment, including an on-demand environment providing multi-tenant database storage services. Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 500 includes one or more processors 502, a main memory 504 (e.g., read-only memory (ROM), flash memory, dynamic random access memory (DRAM) such as synchronous DRAM (SDRAM) or Rambus DRAM (RDRAM), etc., static memory 542, such as flash memory, static random access memory (SRAM), volatile but high-data rate RAM, etc.), and a secondary memory 518 (e.g., a persistent storage device including hard disk drives and persistent multi-tenant data base implementations), which communicate with each other via a bus 530. Main memory 504 includes instructions 524 (such as software 522 on which is stored one or more sets of instructions 524 embodying any one or more of the methodologies or functions of material mechanism 110 of computing device 100 of FIG. 1 and other figures described herein) which operate in conjunction with processing logic 526 and processor 502 to perform the methodologies discussed herein.

Processor 502 represents one or more general-purpose processing devices such as a microprocessor, central processing unit, or the like. More particularly, the processor 502 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 502 may also be one or more special-purpose processing devices such as an application specific integrated circuit (ASIC), a field programmable gate array (FPGA), a digital signal processor (DSP), network processor, or the like. Processor 502 is configured to execute the processing logic 526 for performing the operations and functionality of material mechanism 110 of computing device 100 of FIG. 1 and other figures discussed herein.

The computer system 500 may further include a network interface device 508, such as a network interface card (NIC). The computer system 500 also may include a user interface 510 (such as a video display unit, a liquid crystal display (LCD), or a cathode ray tube (CRT)), an alphanumeric input device 512 (e.g., a keyboard), a cursor control device 514 (e.g., a mouse), a signal generation device 540 (e.g., an integrated speaker), and other devices 516 like cameras, microphones, integrated speakers, etc. The computer system 500 may further include peripheral device 536 (e.g., wireless or wired communication devices, memory devices, storage devices, audio processing devices, video processing devices, display devices, etc.). The computer system 500 may further include a hardware-based application programming interface logging framework 534 capable of executing incoming requests for services and emitting execution data responsive to the fulfillment of such incoming requests.

Network interface device 508 may also include, for example, a wired network interface to communicate with remote devices via network cable 523, which may be, for example, an Ethernet cable, a coaxial cable, a fiber optic cable, a serial cable, a parallel cable, etc. Network interface device 508 may provide access to a LAN, for example, by conforming to IEEE 802.11b and/or IEEE 802.11g standards, and/or the wireless network interface may provide access to a personal area network, for example, by conforming to Bluetooth standards. Other wireless network interfaces and/or protocols, including previous and subsequent versions of the standards, may also be supported. In addition to, or instead of, communication via the wireless LAN standards, network interface device 508 may provide wireless communication using, for example, Time Division, Multiple Access (TDMA) protocols, Global Systems for Mobile Communications (GSM) protocols, Code Division, Multiple Access (CDMA) protocols, and/or any other type of wireless communications protocols.

The secondary memory 518 may include a machine-readable storage medium (or more specifically a machine-accessible storage medium) 531 on which is stored one or more sets of instructions (e.g., software 522) embodying any one or more of the methodologies or functions of material mechanism 110 of FIG. 1 and other figures described herein. The software 522 may also reside, completely or at least partially, within the main memory 504, such as instructions 524, and/or within the processor 502 during execution thereof by the computer system 500, the main memory 504 and the processor 502 also constituting machine-readable storage media. The software 522 may further be transmitted or received over network 520 via the network interface card 508. The machine-readable storage medium 531 may include transitory or non-transitory machine-readable storage media.

Portions of various embodiments may be provided as a computer program product, which may include a computer-readable medium having stored thereon computer program instructions, which may be used to program a computer (or other electronic devices) to perform a process according to the embodiments. The machine-readable medium may include, but is not limited to, floppy diskettes, optical disks, compact disk read-only memory (CD-ROM), and magneto-optical disks, ROM, RAM, erasable programmable read-only memory (EPROM), electrically EPROM (EEPROM), magnet or optical cards, flash memory, or other type of media/machine-readable medium suitable for storing electronic instructions.

Modules 544 relating to and/or include components and other features described herein (for example in relation to material mechanism 110 of computing device 100 as described with reference to FIG. 1) can be implemented as discrete hardware components or integrated in the functionality of hardware components such as ASICS, FPGAs, DSPs or similar devices. In addition, modules 544 can be implemented as firmware or functional circuitry within hardware devices. Further, modules 544 can be implemented in any combination hardware devices and software components.

The techniques shown in the figures can be implemented using code and data stored and executed on one or more electronic devices (e.g., an end station, a network element). Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals). In addition, such electronic devices typically include a set of one or more processors coupled to one or more other components, such as one or more storage devices (non-transitory machine-readable storage media), user input/output devices (e.g., a keyboard, a touchscreen, and/or a display), and network connections. The coupling of the set of processors and other components is typically through one or more busses and bridges (also termed as bus controllers). Thus, the storage device of a given electronic device typically stores code and/or data for execution on the set of one or more processors of that electronic device. Of course, one or more parts of an embodiment may be implemented using different combinations of software, firmware, and/or hardware.

Any of the above embodiments may be used alone or together with one another in any combination. Embodiments encompassed within this specification may also include embodiments that are only partially mentioned or alluded to or are not mentioned or alluded to at all in this brief summary or in the abstract. Although various embodiments may have been motivated by various deficiencies with the prior art, which may be discussed or alluded to in one or more places in the specification, the embodiments do not necessarily address any of these deficiencies. In other words, different embodiments may address different deficiencies that may be discussed in the specification. Some embodiments may only partially address some deficiencies or just one deficiency that may be discussed in the specification, and some embodiments may not address any of these deficiencies.

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements. It is to be understood that the above description is intended to be illustrative, and not restrictive.

What is claimed is:

1. A controlled cavitation apparatus to manufacture medicines or cosmetics, the apparatus comprising:
one or more processors to:
facilitate controlled cavitation dispersion for de-agglomeration of a compound that is agglomerated and represents a mixture of ingredients associated with a medical drug or a cosmetic item; and
generate the medical drug or the cosmetic item based on the controlled cavitation dispersion of the compound.

2. The apparatus of claim 1, wherein one or more processors are further developed to eliminate or at least minimize agglomeration and clumping of the compound through optimization of surface areas and use of nano materials within the compound, wherein the nano materials represent super capacitors or catalytic converters.

3. The apparatus of claim 1, wherein the one or more processors are further to enhance performance of the medical drug or the cosmetic item through uniform particle sizing to enable improved efficacy and potency of the medical drug and the cosmetic item.

4. The apparatus of claim 1, wherein the one or more processors are further to:
receive and evaluate the ingredients associated with the medical drug and the cosmetic item; and
mix the ingredients to form the compound, wherein one or more solvent are added to the compound to ready the compound for one or more treatments.

5. The apparatus of claim 1, wherein the one or more processors are further to:
pass the compound through the one or more treatments including one or more of a thermal stimulation and a mechanical stimulation to enhance amalgamation of the compound;
enhance the compound through one or more mixing processes including one or more of a planetary mixing, an ultrasonic mixing, and a three-ball milling to adjust viscosity and rheology of the compound to get the compound ready for the controlled cavitation dispersion; and
upon generating the medical drug or the cosmetic item, test the medical drug or the cosmetic item; and
upon successful testing of the medical drug or the cosmetic item, package and distribute the medical drug or the cosmetic item.

6. The apparatus of claim 1, wherein the one or more processors comprise one or more of a central processing unit and a graphics processing unit, wherein the central processing unit hosts a first controlled cavitation component, and wherein the graphics processing unit hosts a second controlled cavitation component.

7. A method comprises:
facilitating, by one or more processors of a controlled cavitation device, controlled cavitation dispersion for de-agglomeration of a compound that is agglomerated and represents a mixture of ingredients associated with a medical drug or a cosmetic item; and
generating the medical drug or the cosmetic item based on the controlled cavitation dispersion of the compound.

8. The method of claim 7, further comprising eliminating or at least minimizing agglomeration and clumping of the compound through optimization of surface areas and use of nano materials within the compound, wherein the nano materials represent super capacitors or catalytic converters.

9. The method of claim 7, further comprising enhancing performance of the medical drug or the cosmetic item through uniform particle sizing to enable improved efficacy and potency of the medical drug and the cosmetic item.

10. The method of claim 7, further comprising:
receiving and evaluating the ingredients associated with the medical drug and the cosmetic item; and
mixing the ingredients to form the compound, wherein one or more solvent are added to the compound to ready the compound for one or more treatments.

11. The method of claim 7, further comprising:
passing the compound through the one or more treatments including one or more of a thermal stimulation and a mechanical stimulation to enhance amalgamation of the compound;
enhancing the compound through one or more mixing processes including one or more of a planetary mixing, an ultrasonic mixing, and a three-ball milling to adjust viscosity and rheology of the compound to get the compound ready for the controlled cavitation dispersion; and
upon generating the medical drug or the cosmetic item, testing the medical drug or the cosmetic item; and
upon successful testing of the medical drug or the cosmetic item, packaging and distributing the medical drug or the cosmetic item.

12. The apparatus of claim 7, wherein the one or more processors comprise one or more of a central processing unit and a graphics processing unit, wherein the central processing unit hosts a first controlled cavitation component, and wherein the graphics processing unit hosts a second controlled cavitation component.

13. At least one machine-readable medium having stored thereon instructions which when executed by a processing device, causes the processing device to perform operations comprising:
facilitating controlled cavitation dispersion for de-agglomeration of a compound that is agglomerated and represents a mixture of ingredients associated with a medical drug or a cosmetic item; and
generating the medical drug or the cosmetic item based on the controlled cavitation dispersion of the compound.

14. The machine-readable medium of claim 13, wherein the operations further comprise eliminating or at least minimizing agglomeration and clumping of the compound through optimization of surface areas and use of nano materials within the compound, wherein the nano materials represent super capacitors or catalytic converters.

15. The machine-readable medium of claim 13, wherein the operations further comprise enhancing performance of the medical drug or the cosmetic item through uniform particle sizing to enable improved efficacy and potency of the medical drug and the cosmetic item.

16. The machine-readable medium of claim 13, wherein the operations further comprise:
receiving and evaluating the ingredients associated with the medical drug and the cosmetic item; and
mixing the ingredients to form the compound, wherein one or more solvent are added to the compound to ready the compound for one or more treatments.

17. The machine-readable medium of claim 13, wherein the operations further comprise:
passing the compound through the one or more treatments including one or more of a thermal stimulation and a mechanical stimulation to enhance amalgamation of the compound;
enhancing the compound through one or more mixing processes including one or more of a planetary mixing, an ultrasonic mixing, and a three-ball milling to adjust viscosity and rheology of the compound to get the compound ready for the controlled cavitation dispersion;
upon generating the medical drug or the cosmetic item, testing the medical drug or the cosmetic item; and
upon successful testing of the medical drug or the cosmetic item, packaging and distributing the medical drug or the cosmetic item.

18. The machine-readable medium of claim 13, wherein the processing device comprises one or more of a central processing unit and a graphics processing unit, wherein the central processing unit hosts a first controlled cavitation component, and wherein the graphics processing unit hosts a second controlled cavitation component
